(19) United States
(12) Patent Application Publication
(10) Pub. No.: US 2006/0014271 A1
(43) Pub. Date: Jan. 19, 2006

(54) Fabrication of a completely polymeric microfluidic reactor for chemical synthesis

(76) Inventors: Yujun Song, Baton Rouge, LA (US); Challa Kumar, Baton Rouge, LA (US); Josef Hormes, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 10/893,688

(22) Filed: Jul. 16, 2004

(51) Int. Cl. C12M 1/34; H01L 21/00

(52) U.S. Cl. 435/287.2; 438/1

(57) ABSTRACT

An inexpensive apparatus and method of fabricating completely polymeric (e.g., SU-8, PMMA, and PEEK) microfluidic reactors suitable for the synthesis of chemicals, particularly nanoparticles (e.g., mono, bi, tri, alloy, core-shell, polymeric, and metal-polymer nano-particles), is disclosed. A high precision process uses polymeric microfluidic patterning techniques and a new microfluidic sealing technique, referred to as “flexible semi-solid transfer,” to fabricate high aspect ratio polymeric micro-reactors. In one embodiment, high quality microfluidic channels are patterned onto a support substrate. The microfluidic structure is then sealed by transferring a polymeric material from a sacrificial substrate to the microfluidic structure, and cured. Then, the structure is bonded to a second support structure to form a micro-reactor.

The development of this invention was partially funded by the Government under grant no. NSF/LEQSF (2001-04) RII-03 from the United States National Science Foundation. The Government has certain rights in this invention.

This invention pertains to polymeric micro-reactors, particularly a device and method of fabricating a complete polymeric micro-reactor for manufacturing chemicals such as nano-materials.

Chemical manufactures currently use a technique referred to as “scale-up” to massively produce chemicals using large-size batch reactors. These batch reactors often require large volumes of raw materials and products, which increase complications associated with large-scale transport and storage, and safety and health issues related to potential explosions, and toxin and flammable solvent leakages.

Microfluidic reactors for process scale-up, based on the concept of parallel processing, are increasingly showing potential for controlling the synthetic aspects of the final product to produce chemicals having higher yield and purity. Micro-reactors minimize some of the health and safety risks associated with traditional chemical scale-up processes, by increasing compound reaction efficiency and the controllability of compound reactions, and by reducing the amounts of raw materials and products needed to induce a compound reaction. Micro-reactors also have higher mass and heat transfer efficiency than traditional chemical processes, and may be used, for example, to perform wet chemical synthesis of nanoparticles. See S. J. Haswell, et al., “Micro-chemical Reactors: The Key to Controlling Chemistry,” Royal Society of Chemistry, vol. 250, pp. 25-33 (2000); and P. Watts et al., “Electrochemical Effects Related To Synthesis In Micro-reactors Operating Under Electrokinetics Flow,” Chemical Engineering Journal, vol. 101 (1-3), pp. 237-240 (2004).

Some obstacles associated with process scale-up using microfluidic reactors include, for example, the high costs associated with fabricating microfluidic reactors using existing rapid prototyping techniques, and the incompatibility of materials used in the fabrication process with chemicals produced by microfluidic reactors. Commercial manufacturers of micro-reactors have traditionally used stainless steel, silicon or borosilicate glass to replicate microfluidic reactors, which often involves lengthy and expensive photolithographic processes. These processes are incapable of achieving deep reactive ion etching (“DRIE”) chemistry, and thus reduce the fabrication efficiency of high aspect ratio channels.

There are several obstacles to the successful rapid proto-typing of microfluidic reactors using polymers. One major obstacle involves sealing of microfluidic channels. Another obstacle involves obtaining a strong bond between microfluidic patterns and substrates. Yet another obstacle involves connecting microfluidic reactors with other instruments, such as pumps, collectors, and detectors. These obstacles limit the ability to fabricate simple, low-cost microfluidic reactors using processes such as LIGA, embossing, casting, injection molding, and imprinting. (“LIGA” is a German acronym for “lithography, electrodeposition, and molding.”) See R. J. Jackman et al., “Microfluidic Systems with On-line UV Detection Fabricated in Photodefinable Epoxy,” J. Micromech. Microeng., vol. 11, pp. 263-269 (2001).

In the last few years, research has been very active on low-cost, mass production microfabrication techniques for manufacturing SU-8-based microfluidic reactors due to the superior chemical and mechanical properties of SU-8, in addition to its ease of fabrication using X-ray or UV-based LIGA processes. Complex and multilayered structures are generally produced with relative ease using SU-8 and other materials, such as polymethyl methacrylate (PMMA), polycarbonate (PC), and polydimethylsiloxane (PDMS), that are compatible with standard silicon processing conditions. As compared to other materials currently used to fabricate micro-reactors, such as PDMS and PMMA, SU-8 appears to be more suitable, especially for fabricating reactors having fluidic channels with large depths (up to 500 μm). However, there are several complications to fabricating microfluidic reactors with SU-8. First, sealing the microfluidic channels fabricated in SU-8 without clogging or blockage is not currently possible. Second, the surface tension of a liquid at the edge of the microfluidic pattern during spin-coating prevents the fabrication of a uniform surface pattern. See C. Lin et al., “A New Fabrication Process For Ultra-Thick Microfluidic Microstructures Utilizing SU-8 Photoresist,” J. Micromech. Microeng. vol. 12, pp. 590-597 (2002).

Until recently, there were no methods for sealing SU-8 microfluidic channels without clogging or blockage, nor were there any methods for fabrication of a uniform surface pattern during spin-coating. These problems were addressed in R. J. Jackman et al., “Microfluidic systems with on-line UV detection fabricated in photodefinable epoxy,” J. Micromech. Microeng., vol. 11, pp. 263-269 (2001), which discloses a process for sealing microfluidic channels using SU-8 without any cross-linking, and C. Lin et al., 2002, which discloses the use of a “constant-volume-injection” method to achieve a flat surface for overcoming edge-bead effects. However, these methods require additional process steps, the use of a thin film laminate of SU-8, the precise control of bonding temperatures, and the fabrication of uniform surfaces, which increase microfluidic fabrication costs.

U.S. Pat. No. 6,686,184 describes microfluidic networks and methods for fabricating microfluidic networks having one or more levels of microfluidic channels. In one embodiment, the microfluidic network comprises a polymeric structure having at least first and second non-fluidically interconnected fluid flow paths, wherein at least the first flow path comprises a series of interconnected channels within the polymeric structure.

U.S. Pat. Pub. No. 2003/0150555 and U.S. Pat. No. 6,123,798 describe methods for fabricating polymeric microfluidic devices that incorporate microscale fluidic structures without substantially distorting or deforming the structures. In one embodiment, the microfluidic device comprises a first polymeric substrate having at least a first planar surface with a plurality of channels disposed therein and a second polymeric substrate layer having at least a first planar surface bonded to the first planar surface of the first substrate, wherein the first surface of the second substrate has a lower glass transition temperature than the first surface of the first substrate. In another embodiment, the first planar surface of the second substrate is non-solvent bonded to the first planar surface of the first substrate, wherein the first surface of the second substrate does not substantially project into the plurality of channels.

U.S. Pat. No. 6,645,432 describes microfluidic systems and methods for fabricating complex, discontinuous patterns onto surfaces that can also incorporate or deposit multiple materials onto the surface. In one embodiment, a microfluidic system comprises a polymeric structure having at least first and second non-fluidically interconnected fluid flow paths, wherein at least the first flow path comprises a series of interconnected channels within the polymeric structure. In another embodiment, the microfluidic system comprises a polymeric membrane having a first surface with at least one channel disposed therein, and a polymeric region intermediate the first surface and the second surface. The intermediate region includes at least one connecting channel there-through which fluidically interconnects the channel disposed in the first surface with the channel disposed in the second surface of the membrane.

U.S. Pat. Pub. No. 2002/0108860 describes microfluidic devices and a process for fabricating microfluidic devices comprising emitting microdroplets of a polymeric material from a nozzle onto a substrate, and forming a pattern of microfluidic device features on the substrate using the polymeric material.

An unfilled need exists for a fast and inexpensive microfabrication technique for creating completely polymeric microfluidic reactors for synthesis of chemicals, such as nanoparticles.

We have discovered an inexpensive apparatus and method for microfabrication of complete polymeric (e.g., SU-8, PMMA, and PEEK) microfluidic reactors suitable for the synthesis of chemicals, particularly nanoparticles ranging in size between about 1 nm and about 2000 nm (e.g., mono, bi, tri, alloy, core-shell, polymeric, and metal-polymer nano-particles). This method is a precise process which uses polymeric microfluidic patterning techniques and a new microfluidic sealing technique, referred to as “flexible semi-solid transfer,” to fabricate high aspect ratio polymeric micro-reactors. The novel method provides an improved means for controlling the micro-reactor fabrication process.

In one embodiment, high quality microfluidic channels (e.g., 4-way mixers, multi-pole mixers, and multi-reaction channels) are patterned using SU-8 on a polymeric substrate, such as a PMMA or PEEK substrate. The microfluidic structure is sealed using a thin (about 40-100 μm) SU-8 film coated on a sacrificial substrate, and then exposed to a small dosage (less than about 480 mJ/cm²) of ultraviolet light. After some of the parts of the microfluidic structure are exposed to UV-light through a mask, the unexposed parts of the microfluidic structure are developed away and the structure bonded with PMMA or PEEK to produce a micro-reactor. Embedded structures may be fabricated between the substrates and inlet and outlet channels of the micro-reactor. The micro-reactor may also be combined with an integrated micro heat exchanger or other external or internal components such as micro pumps, valves, and micro separators. Optionally, to further strengthen the completely polymeric micro-reactor, the micro-reactor may be bonded to metallic substrates (e.g., stainless steel, copper, and gold substrates) or ceramic substrates (e.g., alumina and glass substrates).

### DETAILED DESCRIPTION OF THE DRAWINGS

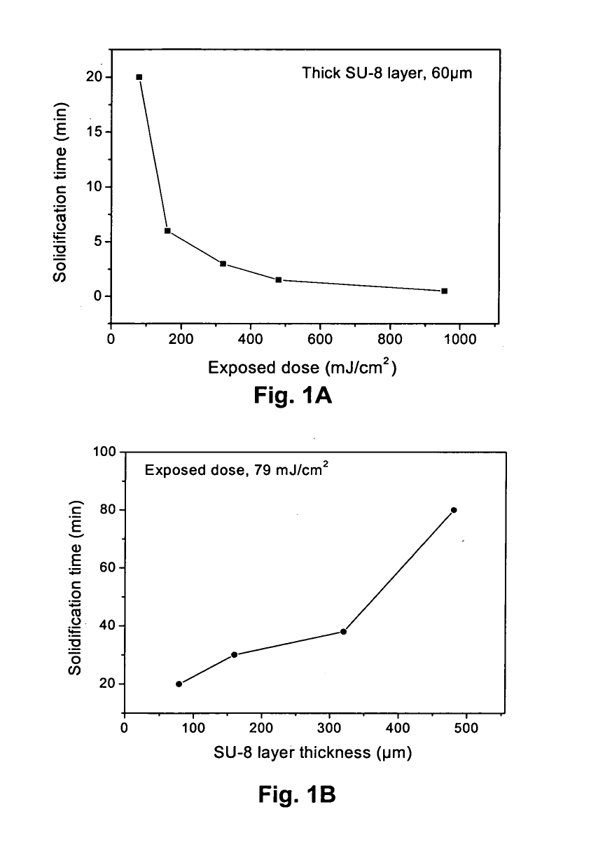

FIG. 1A is a graph plotting the solidification time of a 60 μm thick SU-8 layer as a function of the dose of UV-light exposure.

FIG. 1B is a graph plotting the solidification time of SU-8 exposed to a 79 mJ/cm² dose of UV-light as a function of thickness of the SU-8 layer.

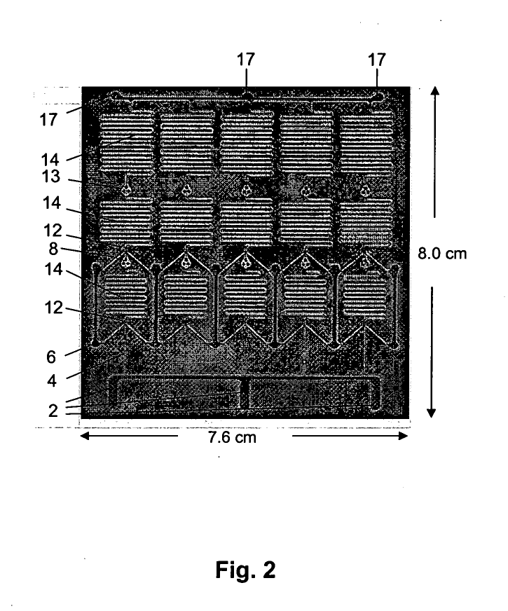

FIG. 2 is an optical micrograph of one embodiment of microfluidic channels.

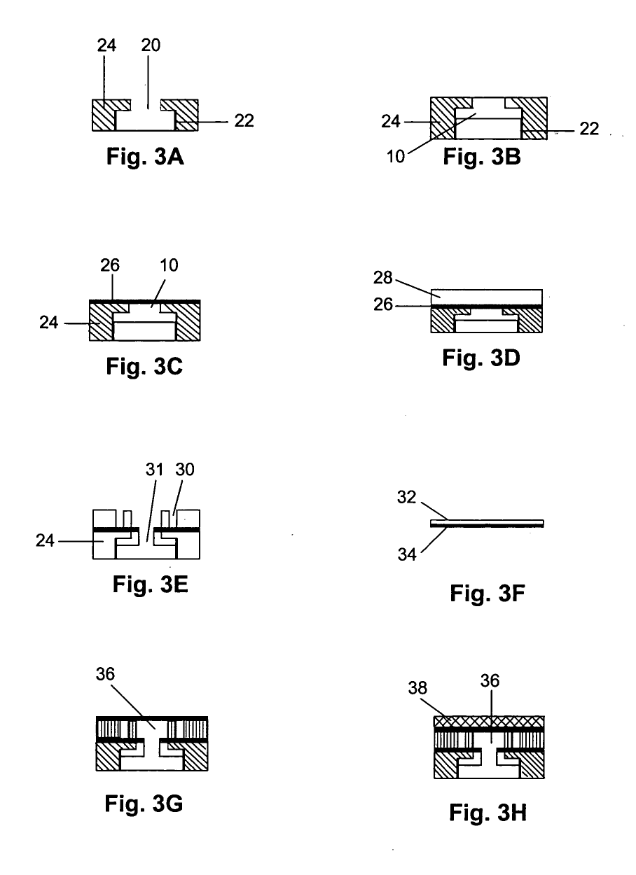

FIGS. 3A-3H illustrate a schematic diagram of a fabrication sequence for the micro-fabrication of one embodiment of microfluidic channels.

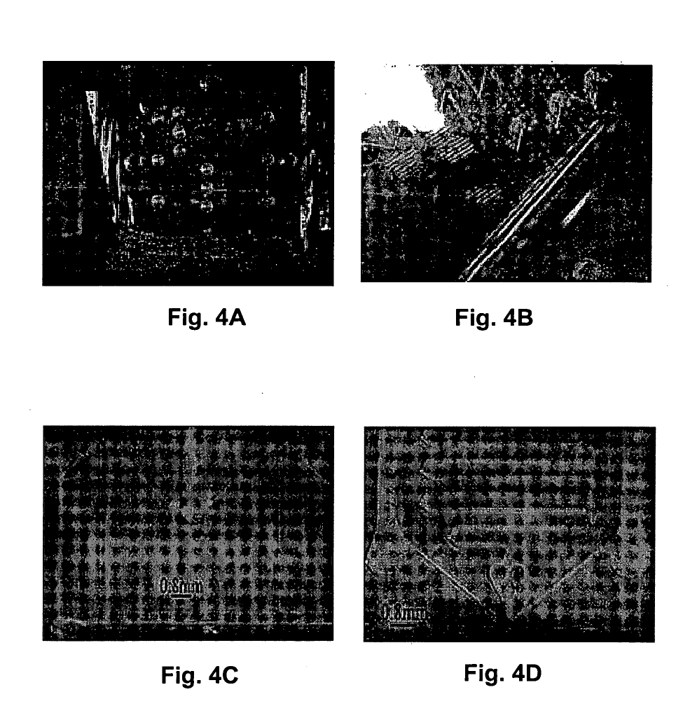

FIG. 4A is an optical micrograph of the patterned microfluidic structure of the micro-reactor covered by a semi-solid SU-8 layer on a polyimide film as shown in FIG. 2.

FIG. 4B is an optical micrograph of the patterned microfluidic structure shown in FIG. 4A after the polyimide was peeled away.

FIG. 4C is an optical micrograph of a 4-way mixer of the microfluidic-patterned micro-reactor shown in FIG. 2 after the microfluidic structure was sealed with a flexible semi solid SU-8 layer.

FIG. 4D is an optical micrograph of the 4-way mixer and a reaction channel of the micro-reactor shown in FIG. 2 after the microfluidic structure was sealed with a flexible semi solid SU-8 layer.

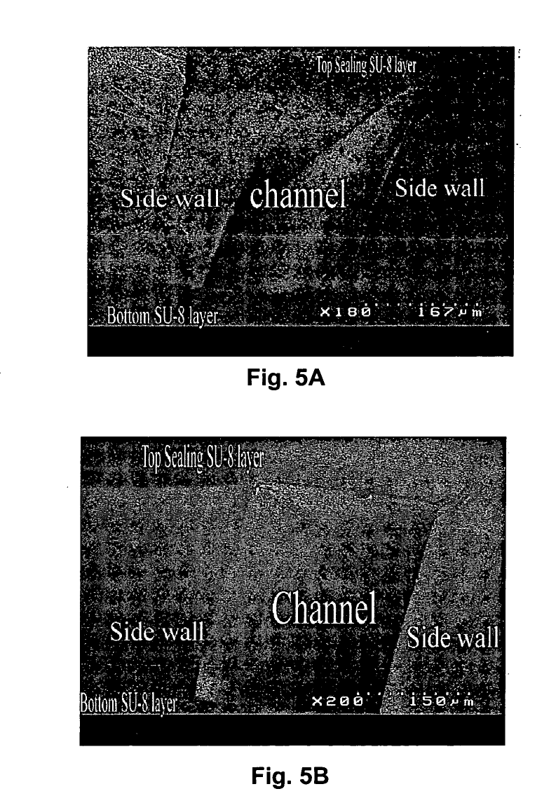

FIGS. 5A and 5B are scanning electron micrographs of inlets to the sealed channels of the micro-reactor shown in FIG. 2.

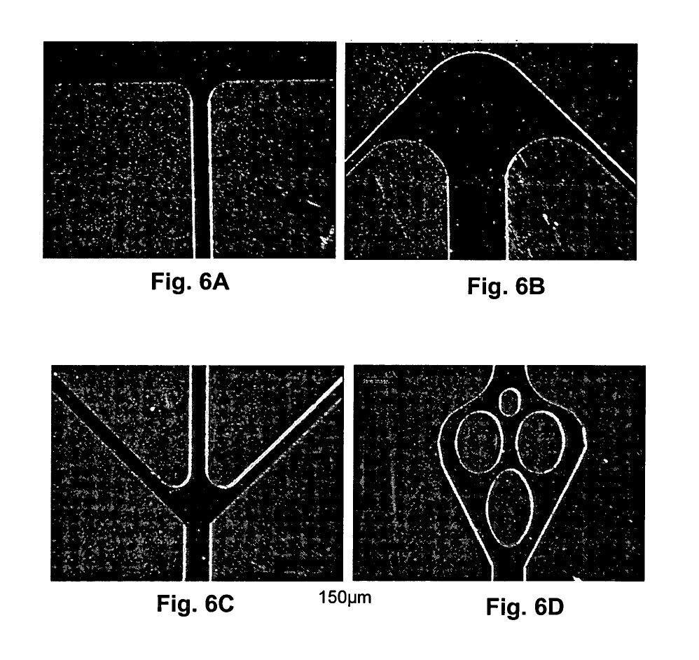

FIGS. 6A and 6B are scanning electron micrographs of inlet channels of the micro-reactor shown in FIG. 2.

FIG. 6C is a scanning electron micrograph (SEM) of a 4-way mixer of the microfluidic-patterned micro-reactor shown in FIG. 2.

FIG. 6D is a scanning electron micrograph of a multi-pole mixer of the microfluidic-patterned micro-reactor shown in FIG. 2.

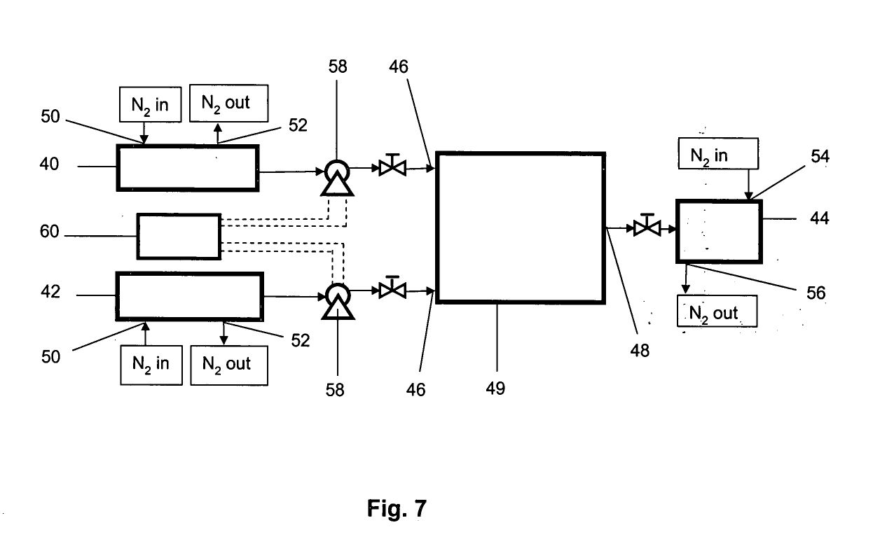

FIG. 7 illustrates a schematic diagram of one embodiment of a continuous flow micro-reactor process for synthesis of palladium nano-particles.

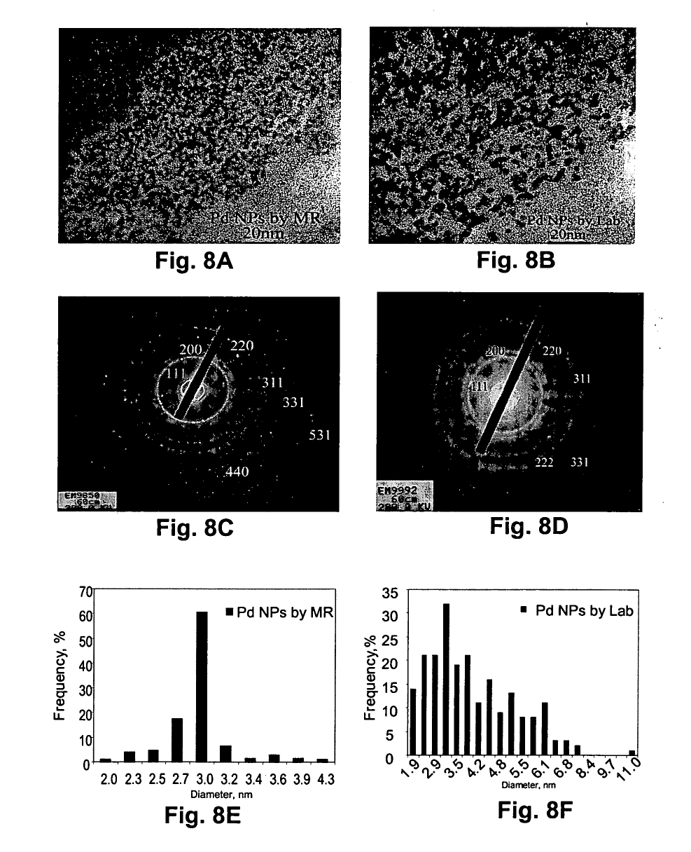

FIG. 8A is a transmission electron micrograph of Pd nanoparticles synthesized using the polymeric micro-reactor shown in FIG. 2.

FIG. 8B is a transmission electron micrograph of Pd nanoparticles synthesized from a conventional batch process.

FIG. 8C is a Selected Area Electron Diffraction pattern (SAED) for the nanoparticles shown in FIG. 8A, which were synthesized using a polymeric micro-reactor process.

FIG. 8D is a SAED pattern for the nanoparticles shown in FIG. 8B, which were synthesized in a conventional batch process.

FIG. 8E is a graph plotting the size distribution of Pd nanoparticles from the polymeric micro-reactor shown in FIG. 2.

FIG. 8F is a graph plotting the size distribution of Pd nanoparticles synthesized from the conventional batch process of FIG. 8B.

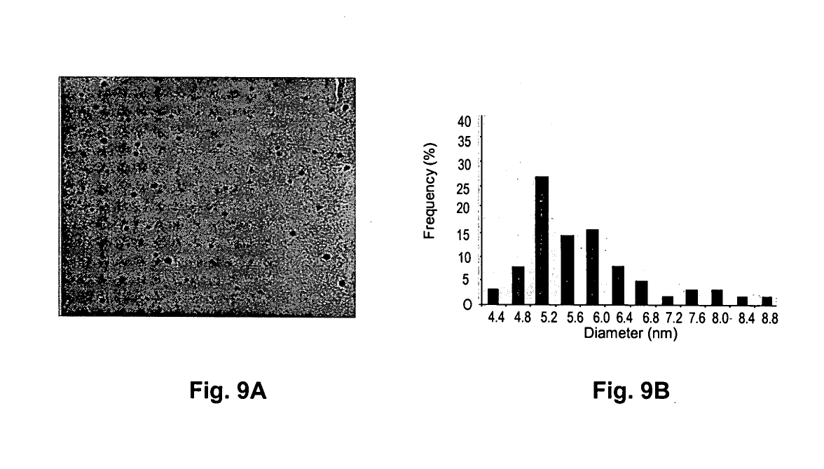

FIG. 9A is a transmission electron micrograph (TEM) of Pd nanoparticles synthesized by one embodiment of the micro-reactor shown in FIG. 2 using a molar ratio of PdCl₂/SB12=2/1.

FIG. 9B is a graph plotting the size distribution of Pd nanoparticles synthesized by the micro-reactor shown in FIG. 3 using a molar ratio of PdCl₂/SB12=2/1.

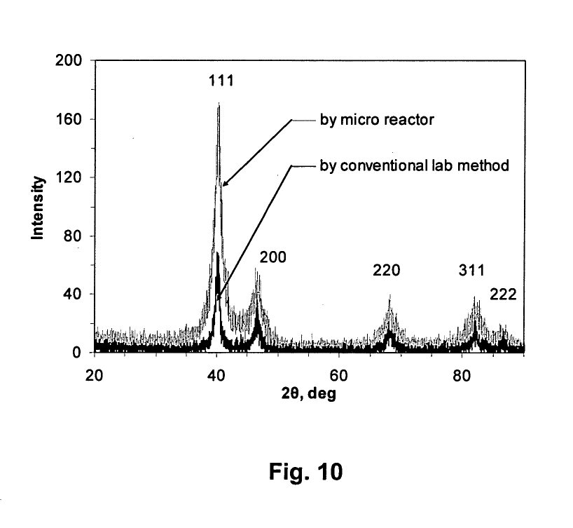

FIG. 10 is a graph comparing the X-Ray Diffraction (XRD) of Pd nanoparticles synthesized by the micro-reactor shown in FIG. 2 and a conventional batch process.

A general purpose of this invention is to provide an apparatus and method for rapid production of completely polymeric microfluidic reactors for chemical synthesis, chemical process development, and process scale-up. More specifically, a purpose of this invention is to provide an inexpensive method for rapid fabrication of completely polymeric microfluidic structures suitable for the synthesis of nanoparticles (e.g., mono, bi, tri, alloy, core-shell, polymeric, and metal-polymer). There are several essentials for facilitating the replication of high quality polymeric micro-reactors. First, the fabrication process should be capable of sealing the microfluidic channels, while avoiding complications associated with clogging and blocking, in addition to avoiding the formation of a non-uniform surface pattern caused by liquid surface tension buildup at the edge of the pattern when using techniques such as spin-coating. Second, the fabrication process should be capable of providing a strong bond between the support substrate and the polymeric microfluidic structures. Finally, the fabrication process should be capable of providing suitable connectors between the microfluidic reactor and the external components such as reactant reservoirs, pumps, and inlets for inert gas, and to ensure leak-proof operations.

High chemical compatibility between materials used to construct the microfluidic structure is preferred. The microfluidic structure should be compatible with various solvents and harsh chemicals such as tetrahydrofuran, toluene, acetone, acids (e.g, HCl), bases (e.g., NaOH) used by commercial chemical manufacturers during synthesis, and it should be stable at temperatures up to 200° C. A preferred microfluidic patterning material is SU-8 (MicroChem Corporation, Newton, Mass.). SU-8 is preferred because it is suitable for fabricating reactors having fluidic channels with large depths (up to 500 μm), and it has superior chemical and mechanical properties in addition to its ease of fabrication using X-ray or UV-based LIGA. SU-8 has a high glass transition temperature range (between about 150° C. and about 220° C.), a high shear modulus (between about 6.26 MPa and about 7.49 MPa), Young's modulus from 2396-2605 MPa at R.T. and 653-1017 MPa at 150° C. The max operation pressure could be as high as 2.1 MPa for this prototype. It also has a low loss tangent (tan δ<<0.001).

Selection of Materials for Constructing the Microfluidic Structure

In determining an effective material for rapidly fabricating inexpensive prototype micro-reactors for chemical synthesis, several polymers (e.g., PMMA, PDMS and SU-8) were tested for chemical stability and compatibility. To determine the chemical stability of each polymer, samples were incubated for three days in test solutions such as THF, Lithium hydrotriethylborate, metal salt (i.e., PdCl₂), THF solution, 50% (v/v) HNO₃, and 50% (v/v) HCl. Afterwards, each polymer sample was inspected for degradation using infrared spectroscopy. THF (99.90% pure packaged under nitrogen), PdCl₂ (99%), lithium hydrotriethylborate (LiBH(C₂H₅)₃) as 1 M solution in THF, 3-(N,N-dimethyldodecylammonium)-propanesulfonate (SB-12), and acetone (reagent anhydrous, water<0.5%, 99.9+%) were purchased from Aldrich Chem. Corp., Milwaukee, Wis. No significant changes were observed for SU-8 in the THF, metal chloride or reducing agent solutions, including lithium hydrotriethylborate (LiBH(C₂H₅)₃) as 1M solution in THF. SU-8 also remained stable for 12 hr in the 50% HCl or HNO₃ solutions. In comparison, PDMS dissolved in less than 4 hr and PMMA swelled within the first two days.

To confirm that SU-8 had a high level of thermal stability, SU-8 films were prepared under different curing conditions and evaluated using Thermo Gravimetric Analysis (TGA) (TA Instrument Inc., New Castle, Del.). The SU-8 films were stable at temperatures up to about 200° C., with an observed weight loss of about two percent. Previous studies have shown that the Glass Transition Temperature (T_{g}) of SU-8 films varies depending on curing conditions, and that SU-8 prepared under standard conditions has a T_{g} of 150° C. Thus, the maximum operating temperature of SU-8-based microfluidic reactors is normally about 150° C. See K. Lian et al. (2003).

Selection of a Suitable Support Substrate for SU-8 Microfluidic Structure

Stainless steel, poly(methyl methacrylate) or PMMA, polyetheretherketone (PEEK), and silicon wafers were considered as a support substrate for the SU-8 microfluidic structure. Silicon wafer was found to be not suitable as a support substrate because it concaved to the side when loaded with an SU-8 multilayer microfluidic pattern due to shrinking stress loads resulting from the cross-linking of SU-8. When a thin layer (4.5 mm) of PMMA was used, the final microfluidic reactor concaved to the side of PMMA. However, when the thickness of the PMMA substrate increased to 12.5 mm, stable structures were obtained. (For PEEK and stainless steel substrates, a 4.5 mm thickness was sufficient.)

While PMMA, PEEK, and stainless steel have good mechanical and machining properties, other important factors for selection of the substrate, include thermal stability of the substrate, thermal expansion coefficient with respect to SU-8, and compatibility with various solvents and harsh chemicals (e.g., tetrahydrofuran, toluene, acetone, acid, and base). Typical thermal expansion coefficients (α), solubility parameters (δ) and T_{g} for SU-8, stainless steel, PMMA, PEEK, tretrahydrofuran (THF), and acetone are listed in Table 1. As shown in Table 1, stainless steel has good organic solvent compatibility and mechanical properties, but is not compatible with cross-linked SU-8, as shown by the differences in their thermal expansion coefficients. (Experiments have shown that even a small defect in an orifice connecting an SU-8 microfluidic structure body with a stainless steel substrate can lead to debonding and cracking along the defect, resulting in leakage.) In contrast, PMMA has good compatibility with cross-linked SU-8 (as shown by the similar δ and α values shown in Table 1), but may be damaged by solvents such as THF and acetone. By comparison, PEEK seems suitable as a support substrate due to its compatibility with SU-8 (as shown by the similar 8 and a values), its ability to maintain a high level of stability with organic solvents (as shown by the differences in 6 between PEEK, THF, and acetone), and its suitability for machining of orifices of different diameters and types.

TABLE 1

| | | Stainless | | | | |
|---|---|---|---|---|---|---|
| | SU-8 | steel | PMMA | PEEK | THF | Acetone |
| δ, (MPa)^{1/2} | 23.0 | — | 19.0 | 21.3 | 18.6 | 19.3 |
| α, ×10⁻⁵/K | 5.7 | 5600 | 5.0 | 5.8 | — | — |
| Tg, ° C. | 150-240 | — | 105-115 | 172-178 | — | — |

Selection of a Suitable Sacrificial Substrate

Tests were conducted to determine if a semi-solid SU-8 film on a flexible sacrificial substrate could be successfully transferred to an SU-8 micro fluidic pattern, and the sacrificial substrate removed after curing the SU-8 film to seal the pattern. Several film substrates such as polyethylene, polytetrafluoroethylene, polycarbonate or printing film, and polyimide film were examined for their utility as a sacrificial substrate. Polyimide was found to be the most suitable film substrate, because liquid SU-8 100 adheres to the polyimide film uniformly, and could be peeled away after the SU-8 cured to a solid state without damaging the microfluidic pattern.

Optimization of the Exposure Dosage and Thickness of SU-8

To obtain a flexible semi-solid SU-8, the time required for solidification of SU-8 at different exposure dosages and its relation to the thickness of SU-8 coating were investigated. FIGS. 1A and 1B show graphs plotting the solidification time of SU-8 as a function of the dose of UV-light exposure and thickness, respectively. FIG. 1A shows a graph plotting the solidification time of a 60 μm thick SU-8 layer as a function of the dose of UV-light exposure. As the exposure dose increased, the solidification time decreased. FIG. 1B shows a graph plotting the solidification time of SU-8 exposed to a 79 mJ/cm² dose of UV-light as a function of thickness. As the SU-8 layer thickness increased, the solidification time increased. An optimum transfer time may be achieved by manipulating the exposure dose and SU-8 thickness. For a 100 μm thick SU-8 layer and an exposure dosage of 300 mJ/cm², the optimum transferring time ranged between about 5-8 min after exposure. (SU-8 may leak into a microfluidic channel if the transferring time is less than 5 min. Conversely, SU-8 tends to lose its flexibility if the transferring time is greater than 8 min).

Increasing the Thermal Stability of SU-8

To increase the thermal stability of the SU-8 microfluidic reactor, the SU-8 layer used in the sealing process was further exposed to UV-light at 300 mJ/cm². A thermo gravimetric analysis of several 100 μm thick SU-8 films, prepared under varying conditions such as (i) exposure of the SU-8 at a standard UV-light dose of 480 mJ/cm² after pre-baking at 65° C. for 20 min and 95° C. for 120 min, and (ii) exposure of the SU-8 at 300 mJ/cm² (without any pre-baking), followed by post-baking at 65° C. for 20 min and 95° C. for 20 min, and exposure at 300 mJ/cm², showed thermal stability beyond 205° C.

### EXAMPLE 1

FIG. 2 shows one embodiment of a polymeric microfluidic reactor. The micro-reactor was fabricated on a chip having a length of 10 cm, a width of 10 cm, and a 7.6 cm by 8.0 cm pattern comprising five interconnected parallel micro-reactors, each having three inlet orifices 2 and five inlet channels 4 for one reactant, twelve inlet orifices 6 and twenty inlet channels 8 for a second reactant, ten 4-way mixers 12, ten multi-pole mixers 13, three meandering reaction channels 14, and three outlet orifices 17 for products. In this embodiment, inlet orifices 2 having a diameter of 1.6 mm supplied a metal salt THF solution (such as PdCl₂) to reaction channels 14 through 4-way mixers 12. Inlet channels 4 were 150 μm wide and 9.5 mm long. The dimensions of reaction chambers 14 were 300 μm wide by 70 mm long, 400 μm wide by 120 mm long and 400 μm wide by 160 mm long, respectively, and had a depth ranging from about 400 μm to about 700 μm. The parallel micro-reactors had a high aspect ratio ranging between about 7 and about 10, and were adapted to increase yield and decrease mixing volume by dividing a first reagent flow stream into five equal reagent flow streams. In this embodiment, the parallel micro-reactors were adapted to allow for the variation of reagent flow in each reagent flow stream from about 120 μL/min to about 2400 μL/min. Mixing is a critical issue in the design of any liquid phase micro-reactor. To preserve chemical homogeneity, it is essential that the entering reagent flow streams rapidly mix (i.e., mix faster than the time scale of the reaction). Four-way mixers 12 were sized and shaped to aid in distributive mixing by flowing a second reagent flow stream supplied by inlet orifices 2 to the first reagent flow stream using inlet channels 4, and allowing the two reagent flow streams to meet at an angle adapted to increase the mixing efficiency while minimizing backflow and pressure drop. Meandering reaction channels 14 and multi-pole mixers 13 were sized and shaped to speed up the growth process of nano-particles in a controlled manner and to prevent the agglomeration or adhesion of these particles on the reactor walls by inducing flow turbulence.

The micro-reactor was fabricated using PMMA (cast-type) and polyetheretherketone (PEEK) support substrates purchased from McMaster-Carr Supply Co., Atlanta, Ga. Other examples of polymeric support substrates which could be used to fabricate the micro-reactor include, SU-8, polypropylene, polyvinyl chloride, polycarbonate, and polyethylene. The patterning of microfluidic channels of SU-8, spin-coated on PMMA, PEEK and stainless steel substrates was carried out using a UV-light (220-450 nm, Model # 85110; Oriel Corporation, Stratford, Conn.). The UV-light dosage required to pattern the microfluidic channels varied up to about 1680 mJ/cm² for a 500 um thick, soft-baked SU-8.

### EXAMPLE 2

FIGS. 3A-3H shows a schematic diagram of a fabrication sequence for the fabrication of the micro-reactor, as otherwise described in Example 1. First, twelve through-holes 20 having a 2 mm dia (only one hole is shown) with ¼ in—28 top threads 22 were pre-machined in a PMMA (cast-type) substrate 24 (McMaster-Carr Supply, Co., Atlanta, Ga.) to form inlet and outlet orifices, as shown in FIG. 3A. (PEEK or stainless steel (SS 304 W/#8 mirror finish) substrates may be used as alternatives to PMMA.) Next, the top surface of each through-hole 20 was sealed with a thin tape (KAPTON® tape; Lanmar Inc., Northbrook, Ill.). Afterwards, the top surfaces of through-holes 20 were filled with approximately 0.03 mL of SU-8 [SU-8 50 (69 wt. % in Gama-butyrolacetone (GBL)] (Micro-Chem, Newton, Mass.), using a 1 mL syringe, until the bottom surfaces of threads 22 were covered to form a first layer 10 as shown in FIG. 3B. Next, first layer 10 was pre-baked in a conventional oven at 65° C. for 20 min, and then at 95° C. for 12 hr to obtain solid SU-8 without cross-linking to seal through-holes 20. Next, the thin tape on the top surface of each through-hole 20 was removed. Next, a transparent mask having a pre-formed pattern was placed over first layer 24 and exposed to UV-light (about 5040 mJ/cm² for a 1 mm thick SU-8 layer), and then post-baked at 65° C. for 20 min and at 95° C. for 20 min without developing to obtain embedded orifice patterns (not shown). This prevented the development of an uneven film around the sealed holes during the subsequent spin-coating process. Next, a 150 μm thick layer of SU-8 [SU-8 25 (63 wt % in GBL)] was spin-coated onto the top surface of the PMMA substrate 24 to form a second layer 26, as shown in FIG. 3C. Second layer 26 was pre-baked and exposed to UV-light, and then post-baked at 65° C. for 20 min and at 95° C. for 20 min without developing. Next, a 400-700 μm thick layer of SU-8 [SU-8 100 (73 wt % in GBL)] (Micro-Chem, Newton, Mass.) was spin-coated onto second layer 26 (SU-8), prebaked at 65° C. for about 10-20 min and at 95° C. for about 7-10 hr in a conventional oven, as shown in FIG. 3D. Next, substrate 24 was cooled to room temperature to form a third layer 28, and then covered with a mask having a microfluidic pattern. Next, third layer 28 was exposed to UV-light, and then post-baked at 65° C. for 20 min and at 95° C. for 20 min. Next, the unexposed SU-8 coated surfaces were developed for about 20-60 min using an SU-8 developer solution to form the microfluidic pattern 30 and a second orifice pattern 31 embedded in substrate 24, as shown in FIG. 3E. (The SU-8 developer is a proprietary solution for developing SU-8 photoresists, and is distributed by the MicroChem, in Newton, Mass.) Orifice pattern 31 may also be formed using a small drill following the formation of the microfluidic pattern in third layer 28.

The multilayer, embedded SU-8 structure was then sealed using the flexible semi-solid transfer process. To achieve this, a 100 μm thick SU-8 film was coated on a sacrificial substrate 32 such as polyimide film (KAPTON®, Lanmar Inc., Northbrook, Ill.) and pre-exposed to a 300 mJ/cm² dosage of UV-light (standard dosage is 480 mJ/cm²) without soft-baking to form a flexible, semi-solid layer 34 of SU-8 film, as shown in FIG. 3F. Semi-solid layer 34 and sacrificial substrate 32 were then placed onto the microfluidic pattern. Afterwards, the sacrificial substrate 32 was flattened uniformly using a rubber blade. Next, semi-solid layer 34 was baked at 65° C. for 20 min (or at room temperature for an extended time) to completely solidify the layer 34, and then the sacrificial substrate (not shown) was removed, as shown in FIG. 3G. Next, the microfluidic pattern was bonded onto a polymeric support substrate 38 (PMMA) to form a micro-reactor 36, as shown in FIG. 3H. Other examples of polymeric support substrates, include SU-8, PEEK, polypropylene, polyvinyl chloride, polycarbonate, and polyethylene. Alternatively, the microfluidic pattern may be bonded onto metallic or glass ceramic substrates, in addition to other support substrates such as stainless steel or alumina.

Screws (not shown) were then threaded into the orifices to tightly position ferrules next to the SU-8 layer on the bottom of the orifices to prevent any leakage. Finally, multi-layer embedded SU-8 structure 36 was bonded to the bottom of the orifices in substrate 24 to prevent the microfluidic structure from cracking or debonding.

### EXAMPLE 3

FIGS. 4A-4D show optical micrographs of one embodiment of a micro-reactor during the sealing process as described in Example 2. FIG. 4A shows one embodiment of the patterned microfluidic structure comprising a pair of 4-way mixers, a pair of 4-pole mixers, and three reaction channels covered by a thin (40-100 μm) semi-solid SU-8 layer on a polyimide film substrate. The polyimide film substrate uniformly adhered to the semi-solid SU-8 layer. The microfluidic pattern, as shown in FIG. 4B, remained undamaged as the polyimide was peeled away. FIG. 4C shows the 4-way mixers after the microfluidic structure was sealed with the flexible semi solid SU-8 layer. FIG. 4D shows the multi-pole mixer and a reaction channel after the microfluidic structure was sealed with the flexible semi solid SU-8 layer. The flexible semi-solid transfer sealing process did not leak SU-8, nor were there any traces of blockage at the inlets and the sealed reaction channels, as shown in FIGS. 5A and 5B. The surface tension of the semi-solid SU-8 was sufficient to prevent SU-8 from leaking. In addition, the mobility and surface tension of the semi-solid SU-8 allowed the formation of a uniform and complete contact surface between the semi-solid SU-8 layer and the surface of the patterned microstructures, and flat, uniform SU-8 film.

The inlet channels, as shown in FIGS. 6A and 6B, had smooth sidewalls, sharp top rims, and abrupt sidewall-to-bottom transitions. The 4-way mixer was free from defects, as shown in FIG. 6C. A minimal amount of deformation and material pickup were observed on the multi-pole mixer, as shown in FIG. 6D.

Table 2 shows the pressure drop of the micro-reactor estimated at different points in the reaction channels using the following equation:

$$P = \frac{{QC}_{fr} L μ}{2 {AD}_{h}^{2}} \qquad ( 1 )$$

See I. Simpson et al., Microfluidics: Applications in Chemical Processing and Analytical Science Proc. Imeche Micro and Nanotechnology (The Thermofluids Dimension, London, 1995). Here Q is flow rate, m³/s; C_{fr} is the friction coefficient for the rectangular cross section where the width w is bigger than the depth d; A is cross section area, m²; Dₕ is the hydraulic diameter calculated by the equivalent diameter of the same cross section area, m; L is length of the channel, m; and μ is viscosity of the feed, Pa·s. For a diluted THF solution, the viscosity and the diffusion coefficient may be treated as the pure THF solvent. The viscosity is 4.856×10⁻³ Pa·s at 25° C. At a combined flow rate of 760 μL/min, the micro-reactor had a pressure drop of 0.028 MPa, and a maximum pressure drop of 2.1 Mpa without any leakage.

TABLE 2

| | | | Retention |
|---|---|---|---|
| Part No. | Length, mm | P^{a}, Pa | time^{b}, sec |
| 2 | 9.5 | 1090 | — |
| 6 | 69.6 | 2995 | 4.4 |
| 7a | 4.4 | 482 | 0.1 |
| 8 | 120 | 3854 | 11.3 |
| 7b | 4.4 | 370 | 0.1 |
| 9 | 160 | 5161 | 15.1 |
| Outer tube, | 100-200 | 61-122 | 79.1-158.2 |
| φ1.6 | | | |

^{a}Capillary pressure drop for a rectangular cross section was used to estimate pressure. The diffusion coefficient of THF was 5.0 × 10⁻⁹ m² · s⁻¹. The viscosity of THF was 4.856 × 10⁻³ Pa · s at 25° C.
^{b}Retention time was calculated based on the average flow rate.

### EXAMPLE 4

To determine the long-term stability of the micro-reactor shown in FIG. 2 at high temperatures, microfluidic reactors on different support substrates were tested. The highest operating temperature for the micro-reactor without any distortions was about 100° C. on PMMA, and about 150° C. on PEEK. No distortions were found.

### EXAMPLE 5

To demonstrate the effectiveness of the micro-reactor to synthesize nanoparticles, comparative tests were conducted using both a conventional batch process and a continuous flow polymeric micro-reactor to synthesize palladium nanoparticles. Palladium nanoparticles were first synthesized with the conventional batch process by reducing PdCl₂ in THF (99.9% pure packaged under nitrogen) using lithium hydrotriethyl borate (LiBH(C₂H₅)₃) as a reducing agent in the presence of 3-(N,N-dimethyldodecylammonium)-propanesulfonate (SB12) by modifying the wet chemical process in the reaction shown below. See H. Bonnemann, et al., “Nanoscale colloidal metals and alloys stabilized by solvents and surfactants: Preparation and use as catalyst precursors,” J. Org. Metal. Chem., vol. 520, pp. 143-162 (1996).

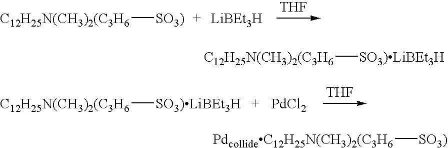

The reaction was conducted under inert atmospheric conditions using the Schlenk technique. The Schlenk technique is used to perform reactions under inert atmospheric conditions. PdCl₂ (0.354 g; 2 mmol) was removed using a 250 mL triple-neck R.B. flask equipped with a flow control inlet adapter, with the flask evacuated completely. Next, the flask was filled with nitrogen and evacuated three times to remove oxygen. Next, 50 mL of THF was added to the reaction flask under nitrogen and the contents stirred magnetically. In a similar fashion, SB12 (0.67 g, 2 mmol) was dissolved under sonication in a 50 mL THF solution containing 4 mmol of lithium hydrotriethyl borate, and then added to a PdCl₂ THF solution drop-wise. Afterwards, the reactants were stirred for an additional 30 min to complete reaction, and then 5 mL of acetone (reagent anhydrous, water<0.5%, 99.9+%) added to destroy reducing agent excess. Next, a 100 mL solution of ethanol (reagent anhydrous, water<0.003%) was added to the reactants, and the Pd nanoparticles were allowed to settle down. Next, supernatant was removed and the particles washed three times using a solution of 50 mL 1:1 volume ratio of THF:ethanol mixture to remove surfactant and other impurities, such as lithium salts. The nanoparticles were then dried using N₂ to obtain a fine black powdery substance. (All of the above-mentioned chemicals were purchased from the Aldrich Chemical Company, Milwaukee, Wis., and used without further purification.)

### EXAMPLE 6

Palladium nanoparticles were then synthesized with the polymeric micro-reactor from Example 1. Reactant reservoirs 40 (PdCl₂ in THF) and 42 (LiBEt₃H in THF) and nanoparticle solution collector 44, as shown in FIG. 7, were connected to inlet orifices 46 and outlet orifice 48 of micro-reactor 49 using ¼ inch—28 fittings and nuts. Reactant reservoirs 40 and 42 were purged by flowing N₂ through inlets 50 and outlets 52. Nanoparticle solution collector 44 was purged by flowing N₂ through inlet 54 and outlet 56. Reactants were pumped into micro-reactor 49 using self-priming pumps 58 (model 120SPI-30; Bio-Chem Valve™ Inc., Boonton, N.J.) with a discrete output of 30 μL/stroke and a set-point accuracy of ±4%. Flow controller 60 comprised a time delay relay (not shown) and a power supply (not shown) to provide self-priming pumps 58 and the time delay relay with 24 DC V. By changing the actuation rate of self-priming pumps 58 from 10 to 200 cycles/min using the time delay relay, the total flow rate ranged from about 600 μL/min to about 12000 μl/min.

FIGS. 8A-8F show the size, size distribution, and crystal structure of nano-particles obtained from the conventional batch process and the polymeric micro-reactor. In both cases, the reducing agent was maintained at a constant flow rate of 380 μL/min. FIGS. 8A and 8B are transmission electron micrograph (TEM) images of Pd nanoparticles obtained from the polymeric micro-reactor and conventional batch process, respectively, which show the difference in particle sizes, size distributions, and shapes. FIGS. 8C and 8D show SAED patterns of Pd nano-particles synthesized by the polymeric micro-reactor and conventional batch process, respectively, for determining crystal structures. FIGS. 8E and 8F show the size distribution plots for Pd nanoparticles obtained from the polymeric micro-reactor and conventional batch process, respectively. The Pd nanoparticles obtained in the conventional batch process had a mean diameter of 3.2 nm with a 35% relative standard deviation, as shown in Table 3. In comparison, Pd nanoparticles obtained from the micro-reactor had a mean particle diameter of 3.0 nm and a narrower size distribution, with a relative percentage standard deviation (% STDV) of 10 likely resulting from the separation of nucleation and growth stage. In growth stage, after particles are formed, they can either agglomerate or smaller particles can redissolve and form bigger particles, leading to broader size distributions. In the micro-reactor process, controlled nucleation and growth stage occurred within the microfluidic channels. Non-uniform diffusion-controlled growth was further limited after the product was placed in a flask as a result of the destruction by acetone of any remaining reducing agent. By contrast, in the conventional batch reaction there was an inevitable concentration gradient as reducing agent was added into the bulk PdCl₂ solution, making it impossible to destroy the excess reducing agent until the reaction was completed, which resulted in non-uniform growth time for all nuclei.

Without wishing to be bound by this theory, it appears that the molar ratio of PdCl₂/SB12 surfactant also affected the size of the palladium nanoparticles prepared within the microfluidic channels, as shown in FIG. 9A. When the molar ratio of PdCl₂/surfactant was increased from 1 to 2, the particle size was found to increase to 5.2 nm with a % STDV of 17, as shown in FIG. 9B. In the conventional batch process, Pd nanoparticle size and size distribution increased as the surfactant concentration decreased. By contrast, in the microfluidic reactor, the Pd nanoparticle size increased with a slight increase in size distribution as surfactant concentration decreased.

TABLE 3

| | | dₙ^{a}, | [symbol] ₙ, | % STDEV, | FWHM01^{b}, | |
|---|---|---|---|---|---|---|
| | Samples | nm | nm | % | degree | \overline{a}ᵢ^{c}, Å |
| | By Lab | 3.2 | 3.9 | 34 | 0.76 | 4.206 |
| By | SB12/PdCl₂ = 2/1 | 3.0 | 3.0 | 10 | 1.22 | 4.217 |
| MR | SB12/PdCl₂ = 1/1 | 5.2 | 5.8 | 17 | — | 4.013 |
| | Bulk Pd | — | — | — | — | 3.8908 |

^{a}dn: the most probability diameter; [symbol] n: the number mean diameter;
^{b}FWHM01, the full-width at half maximum intensity for the most intensive peak;
^{c}\overline{a}ᵢ: the mean lattice parameter calculated from SAED patterns.

An electron diffraction image analysis showed that Pd nanoparticles obtained from the micro-reactor were face-centered cubic (fcc) crystals with a lattice parameter of 4.217 Å, similar to those obtained from the conventional batch process. Compared with the bulk Pd foil, the lattice constants in sulfobetaine-stabilized Pd nanoparticles increased due to the nano size effect. The x-ray diffraction (XRD) pattern of Pd nanoparticles, as shown in FIG. 10, confirmed the fcc structure of the particles in agreement with the electron diffraction analysis. Rings spanning from the inner to the outer direction indicated the crystal plane of 111, 200, 220 and 311 in SAED patterns that were similar to the peaks from the small 2θ angle to large 2θ angle in XRD patterns. (The full-width at half maximum intensity (FWHM) of the first peak (111) and the lattice parameters calculated from the diffraction pattern are shown in Table 3.) The lattice parameters indicated that the micro-reactor achieved a closer packing of atoms in the nanoparticles. The difference of FWHM between the conventional batch process and micro-reactor was consistent with the volume average particle size or the grain size, which implied that the agglomeration of particles in the conventional batch process was more intense than in the micro-reactor.

The peak widths for Pd nanoparticles from the micro-reactor resulted from the crystal size and micro strain effect caused by hydrodynamic forces occurring along the flow orientation in the micro-reactor. These results indicate that there was a slow growth of nanoparticles after a sudden formation of clusters from the saturation solution in the micro-reactor followed by the prevention of Ostwald ripening (i.e., the growth of large crystals from those of smaller size) through decomposition of the excess reducing agent which lead to nearly mono disperse nanoparticles.

The complete disclosures of all references cited in this specification are hereby incorporated by reference. Also incorporated by reference is the following publication of the inventors' own work: Y. Song et al., “Synthesis of Palladium Nanoparticles Using a Continuous Flow Polymeric Micro-reactor,” in Proceedings of the ICCE-10 Symposium on Composites/Nano Engineering, pp. 687-688, held in New Orleans, La. on Jul. 20, 2003; Y. Song et al., “Fabrication of a SU-8 Based Micro Fluidic Reactor on a PEEK Substrate Sealed by a ‘Flexible Semi-solid Transfer’(FST) Process,” J. Micromech. Microeng., vol. 14, pp. 932-940 (2004), and Y. Song et al., “Synthesis of Palladium Nanoparticles Using a Continuous Flow Polymeric Micro Reactor,” Nanosci. and Nanotech., vol. 4, No. 7, pp. 1-6 (2004). In the event of an otherwise irreconcilable conflict, however, the present specification shall control.

We claim:

1. A method for the production of a completely-polymeric micro-reactor, said method comprising the steps of:
(a) fabricating one or more through-holes in a first polymeric support substrate;
(b) sealing the through-holes with a polymeric photo-resist;
(c) forming a first orifice pattern in the polymeric photo-resist located within the through-holes; wherein the first orifice pattern is adapted to form one or more first inlet and outlet orifices;
(d) fabricating a first layer with a second orifice pattern on the first polymeric support substrate, and curing the first layer without developing; wherein the second orifice pattern is sized and shaped to form one or more second inlet and outlet orifices;
(e) coating the first layer with polymeric photo-resist to form a second layer;
(f) fabricating a microfluidic pattern in the second layer, and developing the first and second layers to form one or more embedded microfluidic structures in the first polymeric support substrate; wherein the microfluidic structures are selected from the group consisting of four-way mixers, multi-pole mixers, multi-reaction channels, inlet channels, and outlet channels; wherein the microfluidic structures are adapted to produce nano-particles by the reaction of one or more chemical reactants introduced into the microfluidic structures;
(g) coating a sacrificial substrate with a layer of polymeric photo-resist, and curing the polymeric photo-resist by exposing the polymeric photo-resist to radiation at a dose and time adapted to form a semi-solid microfluidic sealant;
(h) sealing the microfluidic pattern by placing the semi-solid microfluidic sealant on the microfluidic pattern, curing the microfluidic sealant, and removing the sacrificial substrate; and
(i) bonding a second polymeric support substrate to the microfluidic pattern to form a completely-polymeric micro-reactor.

2. A method as recited in claim 1, wherein the polymeric photo-resist comprises SU-8.

3. A method as recited in claim 1, wherein the sacrificial substrate is selected from the group consisting of polyethylene, polytetrafluoroethylene, polycarbonate, polyimide, and printing film.

4. A method as recited in claim 1, wherein the sacrificial substrate comprises polyimide.

5. A method as recited in claim 1, wherein the micro-reactor is adapted to synthesize nano-particles selected from the group consisting of mono, bi, tri, alloy, core-shell, polymeric, and metal-polymer nano-particles.

6. A method as recited in claim 1, wherein the nano-particles comprise palladium nano-particles.

7. A method as recited in claim 1, wherein the first and second polymeric support substrates are selected from the group consisting of polyetheretherketone, poly (methyl methacrylate), SU-8, polypropylene, polyvinyl chloride, polycarbonate, and polyethylene.

8. A method as recited in claim 1, wherein the first and second polymeric support substrates comprise polyetheretherketone.

9. A method as recited in claim 1, wherein the first and second polymeric support substrates comprise poly (methyl methacrylate).

10. A method as recited in claim 1, wherein the micro-reactor is adapted to be fluidically-connected to external components selected from the group consisting of reactant reservoirs, pumps, inlets for inert gas, and micro heat exchangers.

11. A completely-polymeric micro-reactor produced by the method of claim 1.

12. An apparatus for synthesizing nano-particles, comprising:
(a) a first polymeric support substrate comprising one or more through-holes, one or more polymeric photo-resist layers, and a semi-solid sealant layer; wherein said through-holes comprise a first orifice pattern adapted to form one or more first inlet and outlet orifices; wherein at least one of said one or more polymeric photo-resist layers comprises a second orifice pattern adapted to form one or more second inlet and outlet orifices, and wherein at least one of said one or more polymeric layers comprises a microfluidic pattern; wherein said one or more polymeric layers are adapted to interconnect said first inlet and outlet orifices and said second inlet and outlet orifices, and to form one or more embedded microfluidic structures selected from the group consisting of four-way mixers, multi-pole mixers, multi-reaction channels, and inlet channels; wherein said microfluidic structures are adapted to produce nano-particles by the reaction of one or more chemical reactants introduced into the micro-reactor; and wherein said semi-solid sealant layer is adapted to seal said embedded microfluidic structures; and
(b) a second polymeric support substrate; wherein said second polymeric support substrate is bonded to said microfluidic pattern to form a completely polymeric micro-reactor.

13. An apparatus as recited in claim 12, wherein said polymeric photo-resist comprises SU-8.

14. An apparatus as recited in claim 12, wherein said sacrificial substrate is selected from the group consisting of polyethylene, polytetrafluoroethylene, polycarbonate, polyimide, and printing film.

15. An apparatus as recited in claim 12, wherein said sacrificial substrate comprises polyimide.

16. An apparatus as recited in claim 12, wherein said micro-reactor is adapted to synthesize nano-particles selected from the group consisting of mono, bi, tri, alloy, core-shell, polymeric, and metal-polymer nano-particles.

17. An apparatus as recited in claim 12, wherein the nano-particles comprise palladium nano-particles.

18. An apparatus as recited in claim 12, wherein said first and said second polymeric support substrates are selected from the group consisting of polyetheretherketone, poly (methyl methacrylate), SU-8, polypropylene, polyvinyl chloride, polycarbonate, and polyethylene.

19. An apparatus as recited in claim 12, wherein said first and said second polymeric support substrates comprise polyetheretherketone.

20. An apparatus as recited in claim 12, wherein said first and said second polymeric support substrates comprise poly (methyl methacrylate).

21. An apparatus as recited in claim 12, wherein said micro-reactor is adapted to be fluidically-connected to external components selected from the group consisting of reactant reservoirs, pumps, inlets for inert gas, and micro heat exchangers.

22. A process for synthesizing nano-particles using the apparatus in claim 12, said process comprising the steps of:
(a) introducing one or more chemical reactants and one or more reducing agents into at least one of the inlet orifices;
(b) flowing the chemical reactants and reducing agents through the microfluidic structures to produce nano-particles by inducing molecular diffusion of the chemical reactants; wherein the flow rate of the chemical reactants is adapted to control nucleation and growth of the nano-particles; and
(c) collecting the nano-particles through at least one or more of the outlet orifices.

23. A process as recited in claim 22, wherein the flow rate of the reducing agents is about 380 μL/min.

24. A process as recited in claim 22, wherein the apparatus micro-reactor is adapted to control the size, size distribution, and crystal structures of the nano-particles.

25. A process as recited in claim 22, wherein the nano-particles are selected from the group consisting of mono, bi, tri, alloy, core-shell, polymeric, and metal-polymer nano-particles.

26. A process as recited in claim 22, wherein the nano-particles comprise palladium nano-particles.
